(19) United States
(12) Patent Application Publication
(10) Pub. No.: US 2012/0222185 A1
(43) Pub. Date: Sep. 6, 2012

(54) Shower face shield

(76) Inventor: Laura Margaret Erikson, Las Vegas, NV (US)

(21) Appl. No.: 12/932,714

(22) Filed: Mar. 4, 2011

(51) Int. Cl. A61F 9/00

(52) U.S. Cl. 2/11; 2/15

(57) ABSTRACT

A protective face mask comprising: an eye-protecting shield made essentially from a flexible plastic material and an adjustable metal nose bridge. The eye-protecting shield being dimensioned to cover at least the eye portions of a human wearer, the eye-protecting shield having a perimeter defining a surface facing the wearer's face; providing a substantially watertight and releasable sealing engagement between at least a portion of the perimeter forming an upper edge of the face mask and at least a portion of the skin of the wearer's face, a portion of the lower part of the perimeter of the mask is free from the wearer's skin, and wherein the mask extends along the contour of the wearer's face from the forehead to cheekbones and the lower perimeter thereof extends to the bridge of the nose, provides a comfortable and flexible mask without the need for adhesive, straps, or side bars, avoids pressure on the skin in the contact zone, allows free eye movement and airflow and minimizes the formation of dew. The nose bridge is made of a malleable metal material and adjusts to the contours of the wearer's bridge of nose. The mask is primarily adapted for providing a waterproof shield to ensure a dry environment during showering or during trips to the hairdresser, also allowing a person who has recently done their make-up to shower and shampoo their hair while keeping their eye make-up in a clean dry condition.

### BACKGROUND

### Prior Art

The following is a tabulation of some prior art that presently appears relevant:

US. Patents/Patent Application Publication

| Patent/Pub. No. | Kind Code | Issue/Pub. Date | Patentee |
|---|---|---|---|
| 1,640,676 | | 1927-08 | Shaeffer |
| 2,033,691 | | 1936-03 | Douglass |
| 3,310,812 | | 1967-03 | Gaisser |
| 3,545,436 | | 1970-12 | Holloway |
| 4,856,535 | | 1989-08 | Forbes |
| 5,685,022 | A | 1997-11 | Essman |
| 5,865,196 | A | 1999-02 | Foote |
| 5,857,218 | A | 1999-01 | Kuhlman |
| 6,213,125 | B1 | 2001-04 | Reese |
| 7,275,819 | B2 | 2007-10 | Bleau |
| 7,774,858 | B2 | 2010-08 | Kayerod |
| US 2010/0299815 | A1 | 2010-12-02 | Wells |

Foreign Patent Documents

| Foreign Doc. Nr. | Cntry Code | Kind Code | Pub. Dt | Patentee |
|---|---|---|---|---|
| 110147 | DE | | 2005-11 | Edinger |

### DESCRIPTION

The present invention relates to a face mask comprising a protective eye shield dimensioned to cover at least the eye portions of a human wearer. The face mask is particularly useful for shielding against water or shampoo during showering and hair washing. The face mask is also applicable in hairdressing for protecting the wearer's eyes from hair tonic, rinse, hair spray, hair color and clippings.

### BACKGROUND OF THE INVENTION

1. Field of the Invention

Eye goggles and face masks are habitually used to keep the wearer's eyes protected from external environments. For example, swimming goggles are used to protect the wearer's eyes from water. It is important that a face mask properly fit the face of the wearer. The need for a good seal is self-evident, as is the importance of comfort in the fit of the mask. If the mask is not comfortable to wear, it will bother the user and may create feelings of uneasiness. The present invention relates to a light weight protective shield to cover and protect the eyes and a portion of the face of the wearer during showers, hair washing, or hairdressing to maintain a dry environment.

2. Description of the Related Art

In the past it has been necessary to direct the spray of water during showering and shampooing in such a manner to avoid getting water in the eyes. This is of course a difficult task and it is not always possible to keep the entire facial area completely dry due to the overspray of the shower nozzle. There is thus a need for a device which will effectively and comfortably seal against the user's forehead and face to ensure a dry environment during showering or during trips to the hairdresser. There is a need for person's who do not want to have their eye makeup altered to keep eyes completely dry while showering. Also, there are person's for medical reasons such as post surgical procedures or eye infections who cannot allow any liquids, shampoos, dyes or cleaning agents to enter into the eyes during the cleaning/treatment of the scalp/hair.

Traditional eye goggles and face masks normally comprise a spongy or rubber-like material for providing a seal between a perimeter of the goggles or face masks and a portion of the face surrounding the wearer's eyes. Such goggles and face masks are typically made of a rigid plastic material. This rigid plastic material can make the goggles uncomfortable for the wearer. The eye goggles or face masks may be held in place, firmly engaging the wearer's face, by adhesive, side bars, or elastic bands. Goggles and face masks are traditionally sealed around the entire eye contour of the wearer. Goggles that seal around the entire eye area will sometimes collect dew on the inside of the mask making it difficult for the user to see. Goggles that seal with an adhesive are not reusable and can leave a sticky residue on the skin. Goggles with side bars can put undue pressure on side of head causing headaches. Goggles with an attached strap can be bothersome when trying to shampoo hair.

U.S. Pat. No. 4,856,535 discloses a shield for providing a water proof seal across the forehead and along the sides of the face shield to allow a patient having recently had facial cosmetic or reconstructive surgery to shower and shampoo their hair while keeping the bandages and incisions in a clean dry condition. The edge of the shield having the adhesive is pressed against the patient's forehead adjacent the hairline and down along the temples and the side of the face. The shield is of a flexible material allowing the bending and forming along the contour of the face while being rigid enough to retain its shape and form a shield spaced from the patient's face, and produces a tent-like structure leading away from the forehead downwardly over the nose and extending beyond the chin of the patient. Since the shield is secured to the user with adhesive, unfortunately, it can only be used once. Also an undesirable sticky residue is left on the surface of the face from the adhesive. As the shield disclosed in U.S. Pat. No. 4,856,535 extends beyond the chin of the patient humidity from exhaled air will condense at the inner surface thereof and form dew being highly undesirable, especially if used for protecting children's eyes, nose and mouth during bathing. Furthermore, children may feel isolated behind a full face mask covered with dew. U.S. Pat. No. 5,857,218 by Kuhlman, comprises of a protective visor which covers the face and eyes. This visor does not offer full eye protection, thus the possibility of leaks of liquids, shampoos, and chemicals may enter into the eyes. U.S. Pat. Application Pub. No. US 2010/0299815 by Wells, demonstrates goggles that attach around each ear of the wearer. These goggles would be uncomfortable to wear as they would pull, possibly causing unnecessary strain on the ears and are meant to be used at the hairdresser with the user leaning his or her head back. Wearing these goggles in the shower would be difficult as they would likely slip down due to the method of attachment.

There is thus a need for a device which is capable of being comfortably worn, is reusable, and is capable of forming an effective water tight seal to cover and protect the desired areas of face and eyes. There is further need for a waterproof face shield, which is inexpensive, light-weight and easy to apply and remove without hindering the ability of the wearer to shower and shampoo effectively and comfortably.

### SUMMARY

It is an object of preferred embodiments of the present invention to provide a comfortable, flexible, and reusable face mask that provides a shield for the wearer's eyes without the need for adhesive, side bars, or strap to keep the face mask in place, said face mask being comfortable and easy to use.

It is a further object of preferred embodiments of the present invention to provide a face mask, which avoids covering the whole face so as not to make the wearer feel anxious and uncomfortable.

Still further it is an object of the invention to provide a face mask which allows for free airflow to the eye region, nose and mouth allowing free breathing through the nose and the mouth, and which minimizes the inconvenience of dewing on the surfaces of the mask and leaves the wearer able to see through the mask.

A yet further object of the invention is to provide a face mask which in use avoids water flowing under the mask and in to the eyes.

### DETAILED DESCRIPTION OF THE PRESENT INVENTION

In a first aspect the present invention provides a protective face mask comprising: an eye-protecting shield with adjustable nose bridge; the eye-protecting shield is made essentially from a plastics material, the eye-protecting shield being dimensioned to cover at least the eye portions of a human wearer, the eye-protecting shield having a perimeter defining a surface facing the wearer's face; wherein the mask extends along the contour of the wearer's face from the forehead to cheekbones.

The face mask of the invention fulfills the above-mentioned objects and is in the form of a simple and cheap product which is easy to manufacture and use and more comfortable than the masks of the state of the art. It has been found that the problems with condensation are minimized using the mask of the invention. It has been found that the lower edge of the face mask should not extend beyond the bridge of the nose in order not to interfere with exhaled air. It has also been found that the lower part of the face mask should suspend over a portion of the cheek bones to allow for free movement of air, helping to reduce condensation.

In a preferred embodiment of the face mask the lower perimeter thereof does not extend over the nose, rather it rests on the bridge of the nose. Using the adjustable nose bridge the face mask comfortably aligns to the shape of the wearer's nose. This embodiment allows for free breathing through the nose while wearing the mask. This embodiment is preferred for use for e.g. children or for adults closing the mouth when showering.

In preferred embodiments, the eye-protecting shield is made from one single type of material. It will hence be appreciated that the face mask of the invention hence may be more flexible and compliant than the eyewear disclosed in U.S. Pat. No. 7,275,819. Furthermore the eye-protecting shield avoids the problem of formation of dew unlike the face mask disclosed in U.S. Pat. No. 4,856,535.

It should further be understood that the eye-protecting shield may have a thickness of approximately 0.004-1.0 mm.

Embodiments include three components only, i.e. the adjustable nose bridge, adhesive to attach the adjustable nose bridge to the shield, and the single-piece eye-protecting shield thereto provide a simplified construction. Generally, the simple structure of the face mask of the present invention allows it to be manufactured at low cost, while being light, comfortable and easy to use, e.g. for protecting the wearer's eyes during treatment or washing of the wearer's hair.

The face mask is designed to be re-usable.

It is desirable that the protective shield and thus the face mask is flexible and light. The eye-protecting shield should preferably be sufficiently light and flexible to allow it to conform comfortably to the shape and contour of the wearer's face in the region of the wearer's eyes and nose. The face mask, or at least a major portion of the eye-protecting shield, is transparent, allowing the wearer to view through the shield.

The detailed description of the invention is provided primarily for purposes of illustrating the preferred embodiment of the invention. It will be recognized by those skilled in the art that the preferred embodiment is not intended to be limited to the particular structures and method of operation as they may be readily modified. It will be further readily apparent to those skilled in the art that numerous other modifications not mentioned herein can still be made without departing from the spirit and scope of the invention as claimed in the following claims.

### DRAWINGS

### Figures

The invention is disclosed more in detail with reference to the drawings in which

FIG. 1 is a front perspective view of the face mask in accordance with the preferred embodiment thereof;

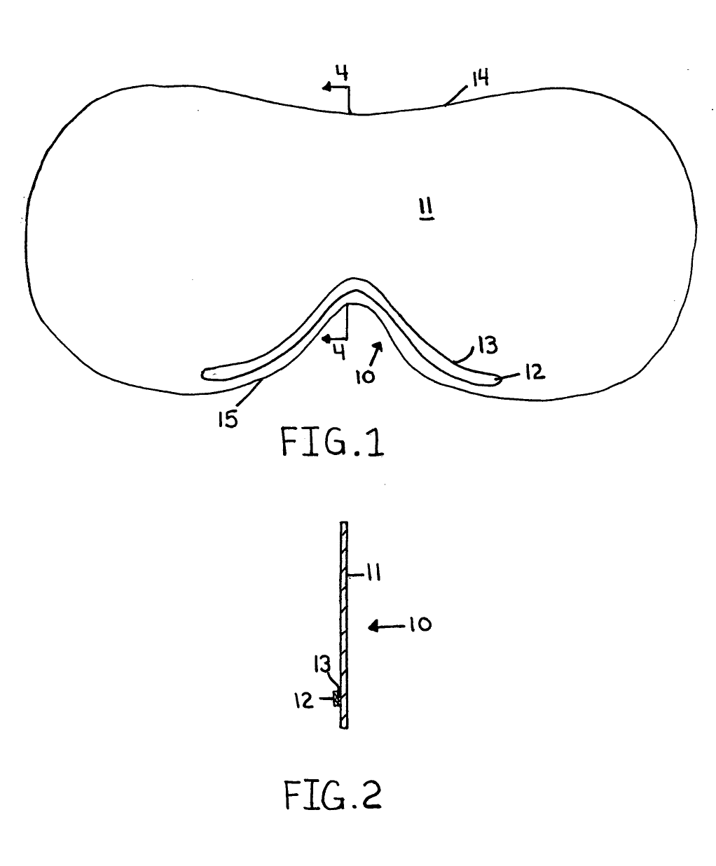

FIG. 2 is a cross sectional view of the face mask as seen along line 4-4 of FIG. 1; and

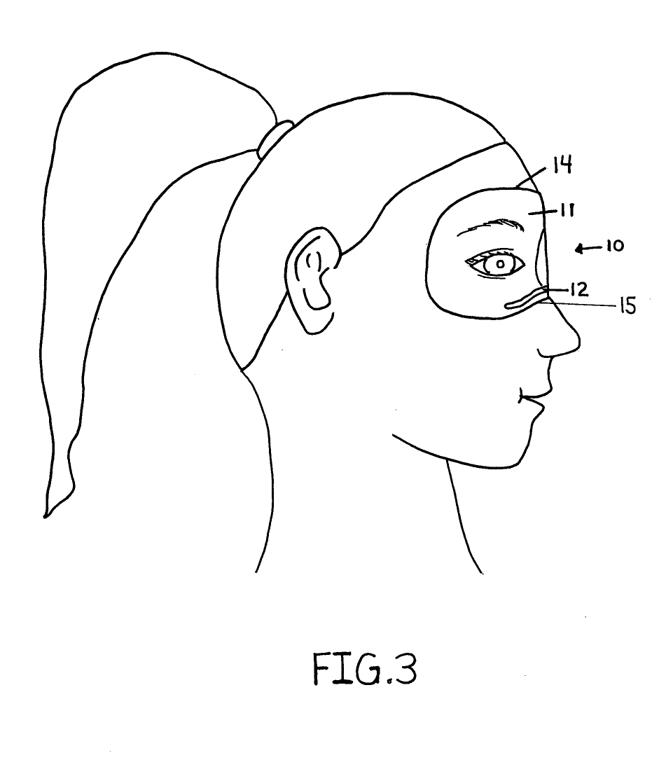

FIG. 3 is a perspective view of the face mask during use as seen from the right side of the wearer.

### DESCRIPTION OF THE PREFERRED EMBODIMENTS

The invention is now explained more in detail with reference to the drawings showing preferred embodiments of the invention but which are not to be construed as limiting the scope of the invention as set forth in the appended claims.
FIG. 1 shows a face mask 10 in accordance with the present invention. The face mask 10 generally provides protection against liquids such as soap and water or rain, and wind without sealing off the entire eye contour and without the use of adhesive, straps, or side bars and furthermore without sealing off the side of the face and the jaw area. The face mask comprises a plastic shield 11 and an adjustable nose bridge 12. At least a portion and preferably the entire plastic shield 11 is transparent. The shield material is flexible and light, so that it easily conforms to the wearer's face. The flexible vinyl material forms a continuous watertight seal between the edge 14 and a portion of the user's face. The adjustable nose bridge 12 is attached near the bottom edge 15 of the plastic shield, is made of a malleable metal such as aluminum, and is shaped to comfortably rest on the bridge of the nose of the wearer.

Suitable materials for the eye-protective shield of the invention are transparent soft flexible materials like e.g. polyvinyl chloride. The shield is preferable a thin, transparent, flexible plastic material which is one dimensional and may be essentially shaped in three dimensions. It is preferably shaped and sized such that the perimeter of the shield extends along with and in contact with the contour of the wearer's face from the forehead to cheekbones and bridge of nose. Other suitable plastic materials are acryl ester materials such as polyurethane film (PU), PMMA, polystyrene materials, polycarbonates or polyolefins such as polyethylene of polypropylene or polyvinyl chloride.

For example, the shield may be made from a single sheet of polyvinyl chloride film, optionally surface-treated.

The entire assembly is maintained on the user's head by simply wetting the upper edge 14 of the face mask and pressing to forehead. Water adhesion between the plastic and skin keep the face mask in place and keep water from entering the wearer's eyes.

FIG. 2 shows a cross sectional view of the face mask as seen along line 4-4 of FIG. 1. The adjustable nose bridge 12 is attached to the face shield 11 by use of an adhesive 13 or similar product.

Suitable materials for the adjustable nose bridge of the invention are bendable metals like e.g. aluminum. The adjustable nose bridge is preferable a thin, malleable metal able to be shaped in three dimensions. Other suitable metals are copper, silver, lead, tin, and aluminum alloys.

FIG. 3 shows a perspective view indicating the size and placement of an embodiment of the face mask 10 of the invention when used in showering.

In FIG. 3, the face mask 10 is illustrated in use. A transparent plastic shield 11 covers the wearer's eyes and is retained in place by water adhesion. FIG. 3 illustrates how the face mask 10 protects the wearer's eyes without sealing off the entire eye contour, providing free eye contour movements and air supply to the eyes. FIG. 3 also illustrates the free access to the user's hair during use of the face mask 10 and also illustrates that the jaw area is free and enables unhindered movement of the jaw, e.g. when speaking.

The invention claimed is:

1. A protective face mask comprising: an adjustable nose bridge and an eye-protecting shield; the eye-protecting shield is made essentially from a plastics material, the eye-protecting shield being dimensioned to cover at least the eye portions of a human wearer, the eye-protecting shield having a perimeter defining a surface facing the forehead and cheekbones of the wearer's face, the eye-protecting shield providing a substantially watertight and releasable engagement between at least a portion of the perimeter forming an upper edge of the face mask and at least a portion of the skin of the wearer's face, the mask extends along the contour of the wearer's face from the forehead to cheekbones and covers the wearer's face from the forehead to bridge of nose.

2. The face mask according to claim 1, wherein the eye-protecting shield is made from a plastics material.

3. The face mask according to claim 1, wherein the eye-protecting shield is flexible, light and transparent.

4. The face mask according to claim 1, wherein the eye-protecting shield is flexible and capable of conforming to the shape and contour of the wearer's face in the region of the wearer's eyes and nose.

5. The face mask according to claim 1, wherein the eye-protecting shield is held in place by water adhesion without the need for straps, adhesive, or side-bars.

6. The face mask according to claim 1, wherein the adjustable nose bridge is made from a malleable metal material.

7. The face mask according to claim 1, wherein the adjustable nose bridge is flexible and capable of conforming to the shape and contour of the wearer's bridge of nose.
